(19) United States
(12) Patent Application Publication
(10) Pub. No.: US 2007/0087327 A1
(43) Pub. Date: Apr. 19, 2007

(54) Method and kit-of-parts for the electrophysiological examination of a membrane comprising an ion channel

(76) Inventors: Niels Fertig, Munchen (DE); Andrea Bruggemann, Munchen (DE)

Correspondence Address: IP STRATEGIES, 12 1/2 WALL STREET, SUITE I, ASHEVILLE, NC, 28801, US

(21) Appl. No.: 11/525,545

(22) Filed: Sep. 22, 2006

(30) Foreign Application Priority Data
Oct. 17, 2005 (EP) 05022618.2

(51) Int. Cl. C12Q 1/00; C12M 3/00

(52) U.S. Cl. 435/4; 435/287.1

(57) ABSTRACT

The invention relates to a collection (kit-of-parts) for producing a high electrical resistance in an electrophysiological examination of a membrane comprising an ion channel, comprising a first electrolytic solution and a second electrolytic solution, wherein the first electrolytic solution comprises 20-140 mM divalent cations of a first element and the second electrolytic solution comprises 20-200 mM monovalent anions of a second element.

The invention relates to a method for the electrophysiological examination of a membrane comprising an ion channel and to a collection (kit-of-parts) for producing a high electrical resistance in an electrophysiological examination of a membrane comprising an ion channel.

Cell membranes (or also artificial lipid membranes) have ion channels, i.e. transmembrane proteins with pores, which allow for a current flow through the membrane. The action of such ion channels can be examined with electrophysiological methods, especially with the patch-clamp technique. In this way, for example, opening and closing mechanisms of the ion channels can be analyzed.

In the conventional patch-clamp method so-called patch-clamp pipettes are used, whereof the aperture diameter at the tip is approximately 1 μm. The shaft of the pipette contains an electrolytic solution (intracellular solution) and an electrode. A membrane patch is sucked onto the aperture of a pipette filled with an electrolytic solution by means of low pressure, so that a close contact is produced between the membrane and the pipette glass. This should now result in a high sealing resistance between the interior of the pipette and the external solution (extracellular solution), in a magnitude of more than one GΩ. In this way, ion channel currents can be measured through the sucked-on membrane patch.

However, this conventional method is not suited for large-scale throughput tests. For such a purpose, however, biochips are known, which have a substrate in which an array of apertures for receiving cell membranes is provided. Such a device is known, for example, from WO 02/066596.

Conventionally used electrolytic solutions for the internal and external solution are described, for example, in A. Ludwig et al., “A family of hyperpolarization-activated mammalian cation channels”, Nature, 1998, Vol. 393, 587-591, A. Brueggemann et al., “Ion Channel Drug Discovery and Research: The Automated Nano-Patch-Clamp Technology”, Current Drug Discovery Technologies, 2004, 1, 91-96 or D. Prawitt et al., “TRPM5 is a transient Ca²⁺-activated cation channel responding to rapid changes in [Ca²⁺]ᵢ”, PNAS, 2003, Vol. 100, No. 25, 15166-15171.

The aforementioned devices provided for the automated performance of patch-clamp methods involve the problem that, with the use of conventional electrolytic solutions, not always a sufficiently high sealing resistance in the magnitude of more than one GΩ is obtained after the membrane patch has been sucked on.

Therefore, it is the object of the invention to provide electrolytic solutions and a method allowing for a high sealing resistance in electrophysiological examinations with improved success.

This object is achieved with a collection according to claim 1 and a method according to claim 10.

According to the invention a collection (kit-of-parts) for producing a high electrical resistance in an electrophysiological examination of a membrane comprising an ion channel is provided, comprising a first electrolytic solution and a second electrolytic solution, wherein the first electrolytic solution comprises 20-140 mM divalent cations of a first element and the second electrolytic solution comprises 20-200 mM monovalent anions of a second element.

Surprisingly, it has been found that a significantly improved production of a sealing resistance is obtained with such a combination of electrolytic solutions in the performance, for example, of a patch-clamp method with a biochip. This particularly occurs if the first electrolytic solution is applied as an extracellular solution (external solution) and the second electrolytic solution as an intracellular solution (internal solution).

The first electrolytic solution may comprise 30-80 mM, especially 30-50 mM, of divalent cations of the first element. Alternatively, or at the same time, the second electrolytic solution may comprise 50-150 mM, especially 60-140 mM, of monovalent anions of the second element. Using electrolytic solutions in these mole ranges results in a further improved resistance production.

The first element may be calcium (Ca) or magnesium (Mg) and/or the second element may be fluorine (F) or chlorine (Cl). Especially, the first electrolytic solution may comprise Ca-ions and the second electrolytic solution may comprise F-ions in the aforementioned amounts of substance.

The divalent cations of the first element may be cations of a chloride salt. Especially, CaCl₂ in the aforementioned amount of substance may be dissolved in the first electrolytic solution.

The monovalent anions of the second element may be fluoride anions. Especially, KF or CsF in the aforementioned amounts of substance may be dissolved in the second electrolytic solution. For example, CaCI₂ may be dissolved in the first electrolytic solution, and KF may be dissolved in the second electrolytic solution. Alternatively, the monovalent anions of the second element may be chloride anions. Therefore, NaCI in the aforementioned amount of substance can be dissolved, for example, in the second electrolytic solution.

The cations and/or the anions may be dissolved in a physiological saline solution, e.g. a Ringer's solution. This permits an examination of cell membranes in their natural environment.

The first and/or the second electrolytic solution may have a pH-value between 7 and 7.5 and/or an osmolarity between 200 and 400 mOsm, especially between 240 and 330 mOsm.

The invention moreover provides for the use of one of the above-described collections for the performance of an electrophysiological examination of a membrane comprising an ion channel, especially for the performance of a patch-clamp method, e.g. of HEK- or CHO-cells.

The collection can especially be used for the performance of patch-clamp method of erythrocytes, primary culture cells or cardiomyocytes. It has been found out that the combination of electrolytic solutions according to the invention also allows patch-clamp examinations of cells, such as erythrocytes, isolated cells/primary culture cells or cardiomyocytes, for which this had otherwise hardly been possible.

In the aforementioned applications, especially the first electrolytic solution can be used as an extracellular solution, and the second electrolytic solution can be used as an intracellular solution.

Moreover, the invention provides for a method for the electrophysiological examination of a membrane comprising an ion channel, especially a cell membrane, comprising the steps:
providing a dividing wall having at least one aperture to receive the membrane,
providing one of the above-described collections,
positioning the membrane on one of the at least one aperture on a first side of the dividing wall, so that the membrane touches the edge of the aperture, wherein the first electrolytic solution is provided on the first side of the dividing wall and the second electrolytic solution is provided on the second side of the dividing wall,
determining the current through or the voltage over the ion channel.

By means of this method a high sealing resistance is produced with great reliability, so that the ion channel current or the voltage over the membrane can be determined with a good signal-to-noise ratio.

The first electrolytic solution can be added after the positioning of the membrane and especially prior to the determination step.

Hence, it is possible, for example, to provide cells to be examined in their original culture medium or an electrolytic solution and to position them at the aperture of the dividing wall. The latter can be accomplished, for example, by applying a low pressure through the aperture and correspondingly sucking in the cell. Only then can the first electrolytic solution be added so that the sealing resistance increases strongly. This also permits the addition of the first electrolytic solution only in case of need, i.e. if the sealing resistance has proved to be too low for the measurements to be performed.

A rinsing may be carried out prior to the determination step to substantially remove the first electrolytic solution.

Surprisingly, it has been found out that the very high sealing resistance achieved by means of the combination of the first and second electrolytic solution according to the invention is also substantially maintained if a rinsing is carried out subsequently. This means that the examination of the membrane can subsequently be performed also with another optional solution without significant changes of the sealing resistance

The dividing wall may be a perforated substrate, especially made of glass or a semiconductor material. With such a substrate the method, especially with a biochip, can be performed in an automated manner, which enables large-scale throughput examinations.

Especially, apertures having a diameter of 0.1-10 μm can be provided with the above-described methods.

Additional advantages and features will be described by means of the figures below.

In the drawings:

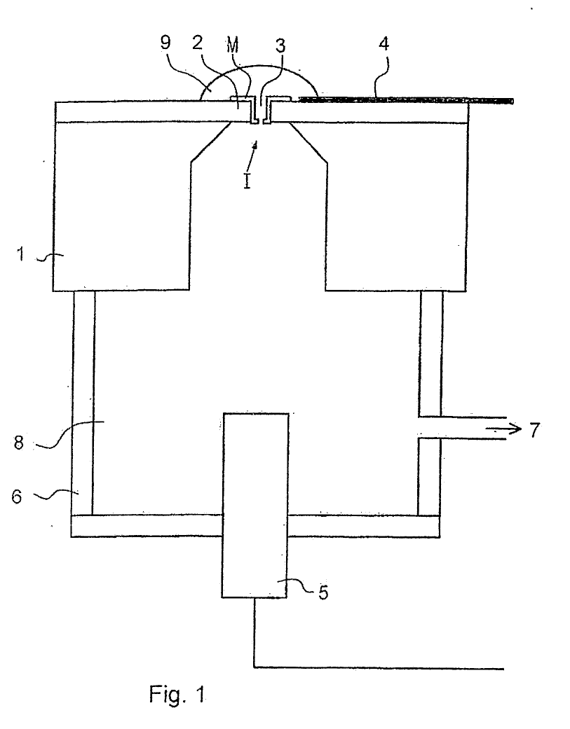

FIG. 1 shows a measuring probe for performing an electrophysiological examination, and

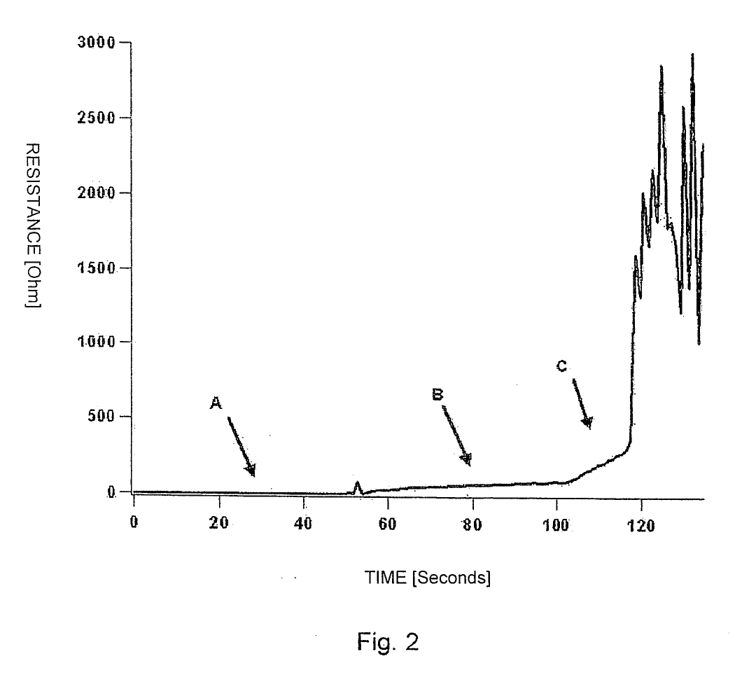

FIG. 2 shows a graphic representation illustrating the increase of the sealing resistance.

The measuring probe shown in FIG. 1 comprises a substrate having a base portion 1 and a window portion 2 in which an aperture 3 is formed. The base portion may be made, for example, of quartz or a semiconductor material, e.g. (100)-Si.

The window portion 2 is formed in an insulating layer made, for example, of glass. The production of such a substrate having a base portion and a window portion is described, for example, in WO 02/066596.

A first electrode 4 is mounted on the substrate. Alternatively, this electrode may also simply be held into the solution without being mounted on the substrate directly. A second electrode 5 is situated underneath the substrate. The electrodes may be made, for example, from Ag/AgCl.

By means of a holding device 6 a cavity is formed, which has an opening with the aperture 3. The measuring probe moreover comprises a device for generating a low pressure in the holding device, as indicated by reference numeral 7.

An intracellular solution 8 is given into the cavity, i.e. underneath the chip, while an extracellular solution 9 is given onto the chip.

Conventional solutions used as intracellular and extracellular solutions are described, for example, in the aforementioned article by A. Ludwig et al. Hence, the extracellular solution can, for example, be composed as follows: 110 mM NaCl, 0.5 mM MgCl₂, 1.8 mM CaCl₂, 5 mM HEPES, 30 mM KCl, adjusted to pH 7.4 with NaOH. An intracellular solution may have the composition: 130 mM KCl, 10 mM NaCl, 0.5 mM MgCl₂, 10 mM EGTA, 10 mM HEPES, adjusted to pH 7.4 with KOH.

The performance of an electrophysiological examination in accordance with the present invention may include, for example, the following steps: Initially, the cavity is filled with an inventive second electrolytic solution as intracellular solution. A suitable intracellular solution can, for example, be composed as follows: 10 mM KCl, 135 mM KF, 10 mM NaCl, 2 mM MgCl₂, 10 mM EGTA, 10 mM HEPES, pH 7.2, 320 mOsm.

Initially, a conventional extracellular solution, described, for example, above in connection with the cited articles, is given onto the chip. Then, the cells or membranes to be examined are added in a conventional extracellular solution. Such a membrane M with an ion channel l is indicated in FIG. 1.

By applying a low pressure the membrane or cell, respectively, is sucked into the aperture 3, which results in an increase of the sealing resistance.

Then, an inventive first electrolytic solution is given onto the chip as extracellular solution, which now leads to the inventive combination of first and second electrolytic solution. The latter results in a significant increase of the sealing resistance. The added electrolytic solution may have the composition: 105 mM NaCl, 4.5 mM KCl, 1 mM MgCl₂, 40 mM CaCl₂, 10 mM HEPES, 5 mM glucose, pH 7.4, 320 mOsm.

The exemplarily mentioned combination of 40 mM divalent Ca-ions in the extracellular solution and 135 mM monovalent F-ions in the intracellular solution results in a great improvement of the electrical resistance over the aperture. By this, patch-clamp examinations can now be performed, especially with a good signal-to-noise ratio.

The variation of the sealing resistance over the time is illustrated in FIG. 2. At the beginning, as long as merely the solutions are provided on either side of the aperture, the resistance of the aperture has a magnitude of approximately 2-3 MΩ (section A). Upon adding the cells the resistance increases to approximately 20-50 MΩ as soon as a membrane is positioned on the aperture. This can be recognized in section B of the graph according to FIG. 2. Till this point the intracellular solution is composed in accordance with the invention, while the extracellular solution is a conventional solution, for example, according to the above description.

However, upon adding the inventive solution (section C) the sealing resistance increases to more than 1 GΩ. This clearly shows that the increase of the sealing resistance is due to the combination of the two solutions according to the invention.

It stands to reason that the above-described exemplary method may also be modified.

Hence, the extracellular solution according to the invention can, for example, be rinsed again after the high sealing resistance was reached, so that the subsequent measurements can be carried out with other conventional extracellular solutions. In this case, too, it shows that the high sealing resistance is maintained.

Furthermore, the surprising effect is not limited to the specific solution compositions mentioned as examples above. For example, divalent Ca- or Mg-ions within the above-specified mole ranges may be dissolved as CaCl₂ or MgCl₂ in a physiological saline solution, e.g. like the Ringer's solution mentioned in Prawitt et al. Correspondingly, also fluoride or chloride may be provided as dissolved in a physiological saline solution, as long as it is within the aforementioned mole range.

Moreover, the invention is not limited to the application of the measuring probe shown in FIG. 1. The collection (kit-of-parts) of the first electrolytic solution and the second electrolytic solution with the indicated mole ranges on divalent cations and monovalent anions can also be applied, for example, in patch-clamp methods with conventional patch-clamp pipettes or large-scale throughput biochips including arrays with a plurality of apertures.

1. A collection (kit-of-parts) for producing a high electrical resistance in an electrophysiological examination of a membrane comprising an ion channel, comprising a first electrolytic solution and a second electrolytic solution, wherein the first electrolytic solution comprises 20-140 mM divalent cations of a first element and the second electrolytic solution comprises 20-200 mM monovalent anions of a second element.

2. A collection according to claim 1, wherein the first electrolytic solution comprises 30-80 mM, especially 30-50 mM, divalent cations of the first element and/or the second electrolytic solution comprises 50-150 mM, especially 60-140 mM, monovalent anions of the second element.

3. A collection according to claim 1, wherein the first element is Ca or Mg and/or the second element is F or Cl.

4. A collection according to claim 1, wherein the divalent cations of the first element are cations of a chloride salt.

5. A collection according to claim 1, wherein the monovalent anions of the second element are fluoride anions.

6. A collection according to claim 1, wherein the cations and/or the anions are dissolved in a physiological saline solution.

7. A collection according to claim 1, wherein the first and/or the second electrolytic solution have a pH-value between 7 and 7.5 and/or an osmolarity between 200 and 400 mOsm, especially between 240 and 330 mOsm.

8. Use of a collection according to claim 1 for the performance of an electrophysiological examination of a membrane comprising an ion channel, especially of erythrocytes, primary culture cells or cardiomyocytes.

9. Use according to claim 8, wherein the first electrolytic solution is applied as extracellular solution and the second electrolytic solution is applied as intracellular solution.

10. A method for the electrophysiological examination of a membrane comprising an ion channel, especially of a cell membrane, comprising the steps:
providing a dividing wall having at least one aperture to receive the membrane, providing a collection according to claim 1,
positioning the membrane on one of the at least one aperture on a first side of the dividing wall, so that the membrane touches the edge of the aperture, wherein the first electrolytic solution is provided on the first side of the dividing wall and the second electrolytic solution is provided on the second side of the dividing wall,
determining the current through or the voltage over the ion channel.

11. A method according to claim 10, wherein the first electrolytic solution is added after the positioning of the membrane.

12. A method according to claim 11, wherein a rinsing is carried out prior to the determination step to substantially remove the first electrolytic solution.

13. A method according to claim 10, wherein a perforated substrate, especially made of glass, is provided as dividing wall.

14. A method according to claim 10, wherein apertures having a diameter of 0.1-10 μm are provided.
